(19) United States
(12) Patent Application Publication
(10) Pub. No.: US 2024/0038341 A1
(43) Pub. Date: Feb. 1, 2024

(54) METHOD, SYSTEM AND STORAGE MEDIUM FOR CONSISTENCY ANALYSIS OF LITHIUM BATTERY MODULE

(71) Applicant: SHANGHAI MAKESENS ENERGY STORAGE TECHNOLOGY CO., LTD., Shanghai (CN)

(72) Inventors: Danfei Gu, Shanghai (CN); Mingchen Jiang, Shanghai (CN); Siyuan Chen, Shanghai (CN); Xiao Yan, Shanghai (CN); Enhai Zhao, Shanghai (CN); Qian Li, Shanghai (CN)

(21) Appl. No.: 18/226,341

(22) Filed: Jul. 26, 2023

(30) Foreign Application Priority Data
Jul. 28, 2022 (CN) 202210900408.9

(51) Int. Cl. G16C 20/30; H01M 10/42; G16C 20/70

(52) U.S. Cl. CPC G16C 20/30; H01M 10/425; G16C 20/70; H01M 2010/4271

(57) ABSTRACT

The invention discloses a method, a system and a storage medium for consistency analysis of a lithium battery module. The method includes acquiring various microscopic electrochemical parameters corresponding to each single cell in the lithium battery module; respectively establishing a time sequence database of the microscopic electrochemical parameters corresponding to each single cell, based on each of the microscopic electrochemical parameters; and performing consistency analysis on each single cell that meets a preset circuit connection relation based on each time sequence database.

### CROSS-REFERENCE TO RELATED PATENT APPLICATION

This application claims priority to and the benefit of Chinese Patent Application No. 202210900408.9, filed Jul. 28, 2022, which are incorporated herein in their entireties by reference.

### FIELD OF THE INVENTION

The invention relates generally to the field of lithium batteries, and more particularly to a method, system, and storage medium for consistency analysis of a lithium battery module.

### BACKGROUND OF THE INVENTION

In the process of establishing an electrochemical model for a lithium battery module, many electrochemical parameters representing current physical and chemical properties of the lithium battery are usually required as parameters for calculation of the electrochemical model of the lithium battery module to simulate operation of the lithium battery module. In order to ensure the accuracy and predictability of the simulation, the consistency of the lithium battery modules participating in the operation simulation needs to be ensured, so that the electrochemical parameters participating in the operation simulation can accurately reflect the current physical and chemical properties of the lithium battery.

In the prior art, consistency analysis of the lithium battery module is performed, based on a voltage time sequence or a temperature time sequence, by using statistical methods. However, in the process of lithium battery consistency analysis, these parameters are only macroscopic external states of the batteries, so the consistency analysis of the lithium battery module cannot truly represent the differences in the internal states of the batteries.

Therefore, there is a need for a method for consistency analysis of lithium battery modules, which, based on the traditional consistency analysis of the electrochemical parameters of the lithium batteries, and through the consistency analysis of more microscopic electrochemical parameters, makes the consistency judgment of the electrochemical parameters of lithium battery modules more intrinsic.

### SUMMARY OF THE INVENTION

In view of the above-noted shortcomings in the prior art, one of the objectives of this invention is to provide a method, a system and a storage medium for the consistency analysis of the lithium battery module to solve the problems in the prior art that the consistency analysis of the lithium battery module is not accurate enough.

In one aspect of the invention, the method for the consistency analysis of the lithium battery module includes acquiring various microscopic electrochemical parameters corresponding to each single cell in the lithium battery module; respectively establishing a time sequence database of the microscopic electrochemical parameters corresponding to each single cell, based on each of the microscopic electrochemical parameters; and performing the consistency analysis on each single cell that meets a preset circuit connection relation based on each time sequence database.

According to the invention, the method for the consistency analysis of the lithium battery module is based on the microscopic electrochemical parameters to establish the time sequence database of the microscopic electrochemical parameters that are corresponding to each single cell in the lithium battery module, and perform the consistency analysis on each single cell that meets the preset circuit connection relation in the lithium battery module according to the microscopic electrochemical parameters, so as to design a consistency analysis scheme from the perspective of the microscopic electrochemical parameters. Compared the consistency analysis of using macroscopic data such as voltages, temperatures and the like, the novel method is more intrinsic.

In some embodiments, the microscopic electrochemical parameters comprise at least one of a solid phase diffusion coefficient, a liquid phase diffusion coefficient, a solid phase conductivity, a liquid phase conductivity, sizes of positive and negative electrode regions, a separator region size, an average particle size, a porous medium coefficient, a solid phase volume coefficient, a liquid phase volume coefficient, an electron transfer coefficient, a solid electrolyte interphase (SEI) film thickness, and a total number of active lithium.

In some embodiments, the microscopic electrochemical parameters include at least one of the SEI film thickness and the total number of active lithium. Said acquiring the microscopic electrochemical parameters corresponding to each single cell in the lithium battery module comprises performing deductive prediction based on a preset electrochemical life decay model, and performing maximum likelihood value estimation of the SEI film thickness or the total number of active lithium after Kalman filtering.

In some embodiments, said performing the consistency analysis on each single cell that meets the preset circuit connection relation based on each time sequence database comprises performing the consistency analysis on each single cell that meets the preset circuit connection relation according to at least one of an outlier frequency, an information entropy, a fluctuation consistency, an angle variance, a density clustering, and a Gini impurity degree of each of the microscopic electrochemical parameter.

In some embodiments, said establishing of the time sequence database of the microscopic electrochemical parameters corresponding to each single cell based on each microscopic electrochemical parameter comprises respectively establishing the time sequence database of the microscopic electrochemical parameters corresponding to each single cell through a cloud server based on each of the microscopic electrochemical parameters according to a preset cloud-edge collaboration scheme.

According to the invention, the method for the consistency analysis of the lithium battery module utilizes a cloud server to carry out operations of establishing the time sequence database of the microscopic electrochemical parameters according to the preset cloud-edge collaboration scheme, so that the technical problem of poor consistency analysis effect caused by insufficient hardware capacity or over-low calculation capacity is solved.

In some embodiments, said acquiring the microscopic electrochemical parameter corresponding to each single cell in the lithium battery module comprises inputting current working condition parameters and current environment parameters of the lithium battery module into a preset lithium battery electrochemical model; obtaining each of the microscopic electrochemical parameters currently corresponding to each single cell in the lithium battery module through parameter identification; and correcting each of the microscopic electrochemical parameters currently corresponding to each single cell in the lithium battery module through a preset machine learning model.

The machine learning model is generated by machine learning, based on the working condition parameters, the environment parameters and the microscopic electrochemical parameters corresponding to each single cell of a set of preset sample lithium battery modules.

According to the invention, the method for the consistency analysis of the lithium battery module utilizes the machine learning to analyze the lithium battery electrochemical model and realizes, by combining artificial intelligence and mechanisms of the lithium battery electrochemical model, the acquisition/collection of the microscopic electrochemical parameters without dismantling the lithium battery module, which not only avoids the errors that occur during measurements of the electrochemical parameters in the traditional methods, thereby affecting the consistency judgment in the lithium battery module, but also improves the intellectualization in the process of the consistency analysis of the lithium battery module.

In some embodiments, said acquiring the microscopic electrochemical parameter corresponding to each single cell in the lithium battery module further comprises generating, by simulation of the lithium battery electrochemical model, each of the microscopic electrochemical parameter corresponding to each single cell in the lithium battery module after a preset time period.

According to the invention, the method for the consistency analysis of the lithium battery module utilizes the lithium battery electrochemical model constructed based on machine learning to predict the trend of the electrochemical parameters of the lithium battery module, and perform the consistency analysis simultaneously according to the present electrochemical parameters and the predicted electrochemical parameters, so that the consistency analysis accuracy of the lithium battery module is improved.

In some embodiments, after performing consistency analysis on each single cell that meets the preset circuit connection relation based on each time sequence database, the method further comprises, when any one of the microscopic electrochemical parameters corresponding to each single cell currently or after the preset time period in the lithium battery module is inconsistent, issuing an early warning.

According to the invention, the method for the consistency analysis of the lithium battery module utilizes the present electrochemical parameters and the predicted electrochemical parameters to carry out the consistency analysis simultaneously and design an early warning scheme, so that the consistency analysis of the lithium battery module is more predictive.

In another aspect of the invention, the system for the consistency analysis of the lithium battery module includes an acquisition module, configured to acquire various microscopic electrochemical parameters corresponding to each single cell in the lithium battery module; a database establishing module connected with the acquisition module and configured to respectively establish a time sequence database of the microscopic electrochemical parameters corresponding to each single cell, based on each of the microscopic electrochemical parameters; and an analysis module connected with the database establishing module and configured to consistency analysis on each single cell that meets a preset circuit connection relation based on each time sequence database

In yet another aspect, the invention relates to a non-transitory tangible computer-readable storage medium storing at least one instruction which, when executed by one or more processors, causes the system to perform the method for consistency analysis of the lithium battery module according as disclosed above.

According to the invention, the method, the system and the storage medium for consistency analysis of the lithium battery module provide at least the following technical effects:
(1) Based on the microscopic electrochemical parameters, the time sequence database of the microscopic electrochemical parameters that are corresponding to each single cell in the lithium battery module is established, the consistency analysis on each single cell that meets the preset circuit connection relation in the lithium battery module is performed according to the microscopic electrochemical parameters, and a consistency analysis scheme is designed from the perspective of the microscopic electrochemical parameters. Compared the consistency analysis of using macroscopic data such as voltages, temperatures and the like, the novel method is more intrinsic.
(2) According to the preset cloud-edge collaboration scheme, a cloud server is adopted to to carry out operations of establishing the time sequence database of the microscopic electrochemical parameters, so that the technical problem of poor consistency analysis effect caused by insufficient hardware capacity or over-low calculation capacity is solved.
(3) The lithium battery electrochemical model is analyzed/deconstructed by using the machine learning, and the acquisition/collection of the microscopic electrochemical parameters is realized/achieved by combining artificial intelligence and mechanisms of the lithium battery electrochemical model, without dismantling the lithium battery module, which not only avoids the errors that occur during measurements of the electrochemical parameters in the traditional methods, thereby affecting the consistency judgment in the lithium battery module, but also improves the intellectualization in the process of the consistency analysis of the lithium battery module.
(4) The trend of the electrochemical parameters of the lithium battery module is predicted by utilizing the lithium battery electrochemical model constructed based on machine learning, and the consistency analysis is simultaneously performed according to the present electrochemical parameters and the predicted electrochemical parameters, so that the consistency analysis accuracy of the lithium battery module is improved.
(5) The consistency analysis is simultaneously carried out by utilizing the present electrochemical parameters and the predicted electrochemical parameters, and an early warning scheme is designed, so that the consistency analysis of the lithium battery module is more predictive.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 is a flowchart of a method for consistency analysis of a lithium battery module according to one embodiment of the invention.

FIG. 2 is a flowchart of consistency analysis performed on each single cell satisfying a preset circuit connection relation in the method for consistency analysis of a lithium battery module according to one embodiment of the invention.

FIG. 3 is a flowchart of establishment of a time sequence database of the microscopic electrochemical parameters corresponding to each single cell based on each microscopic electrochemical parameter in the method for consistency analysis of a lithium battery module according to one embodiment of the invention.

FIG. 4 is a flowchart of acquisition of the microscopic electrochemical parameters corresponding to each single cell in the lithium battery module in the method for consistency analysis of a lithium battery module according to one embodiment of the invention.

FIG. 5 is a flowchart of acquisition of the microscopic electrochemical parameters corresponding to each single cell in the lithium battery module in the method for consistency analysis of a lithium battery module according to another embodiment of the invention..

FIG. 6 is a flowchart of a method for consistency analysis of a lithium battery module according to another embodiment of the invention.

FIG. 7 is an exemplary diagram of a system for consistency analysis of a lithium battery module according to one embodiment of the invention.

### DETAILED DESCRIPTION OF THE INVENTION

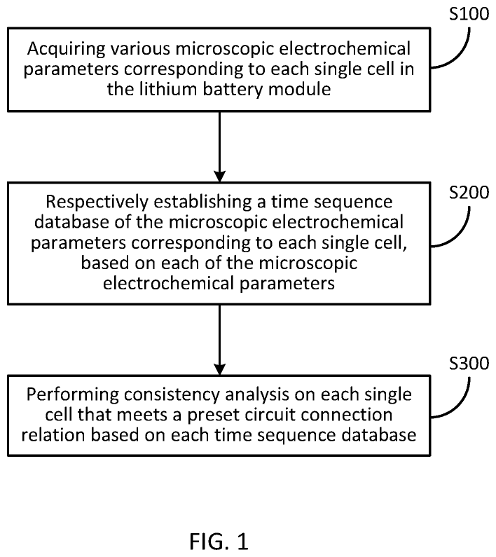

Embodiments of the invention are described below through specific examples in conjunction with the accompanying drawings in FIGS. 1-7, and those skilled in the art can easily understand other advantages and effects of the invention from the content disclosed in this specification. The invention can also be implemented or applied through other different specific implementations, and various modifications or changes can be made to the details in this specification based on different viewpoints and applications without departing from the spirit of the invention. It should be noted that, in the case of no conflict, the following embodiments and features in the embodiments can be combined with each other.

It should be noted that the drawings provided in the following embodiments are merely illustrative in nature and serve to explain the principles of the invention, and are in no way intended to limit the invention, its application, or uses. Only the components related to the invention are shown in the drawings rather than the number, shape and size of the components in actual implementations. For components with the same structure or function in some figures, only one of them is schematically shown, or only one of them is marked. They do not represent the actual structure of the product. Dimensional drawing, the type, quantity and proportion of each component can be changed arbitrarily in its actual implementations. More complicate component layouts may also become apparent in view of the drawings, the specification, and the following claims.

The terminology used herein is for the purpose of describing particular embodiments only and is not intended to be limiting of the invention. As used herein, “a” not only means “only one”, but also means “more than one”. The term “and/or” used in the description of the invention and the appended claims refers to any combination and all possible combinations of one or more of the associated listed items, and includes these combinations. The terms “first”, “second”, etc. 30 are only used for distinguishing descriptions, and should not be construed as indicating or implying relative importance.

It will be understood that the terms “comprises” and/or “comprising,” when used in this specification and the appended claims, specify the presence of stated features, integers, steps, operations, elements, and/or components, but do not preclude the presence or addition of one or more other features, integers, steps, operations, elements, components, and/or groups thereof

Referring to FIG. 1, the method for consistency analysis of a lithium battery module is shown according to one embodiment of the invention. The method includes the following steps.

At step S100, acquiring various microscopic electrochemical parameters corresponding to each single cell in the lithium battery module.

Specifically, the microscopic electrochemical parameters are concepts relative to the macroscopic parameters such as voltage, current and temperature in the prior art, and include, but are not limited to, a solid phase diffusion coefficient, a liquid phase diffusion coefficient, a solid phase conductivity, a liquid phase conductivity, sizes of positive and negative electrode regions, a separator region size, an average particle size, a porous medium coefficient, a solid phase volume coefficient, a liquid phase volume coefficient, an electron transfer coefficient, a solid electrolyte interphase (SEI) film thickness, and/or a total number of active lithium. It should be noted that other electrochemical parameters can also be used to practice the invention.

At step S200, respectively establishing a time sequence database of the microscopic electrochemical parameters corresponding to each single cell, based on each of the microscopic electrochemical parameters.

Specifically, a time sequence database is established for each selected microscopic electrochemical parameter, and the time sequence database comprises time sequence data of the microscopic electrochemical parameter corresponding to each single cell. For example, time sequence data of the solid phase diffusion coefficient corresponding to each of the number 1 to 100 single cells in the lithium battery module is established for the solid phase diffusion coefficient, and is stored in a database corresponding to the solid phase diffusion coefficient, so that the database can be conveniently extracted for consistency analysis.

At step S300, performing consistency analysis on each single cell that meets a preset circuit connection relation based on each time sequence database.

Specifically, the preset circuit connection relationship comprises a series connection, a parallel connection and/or a series-parallel connection, and is determined by manual selection. In the consistency analysis process, the statistical characteristics of the time sequence data of the microscopic electrochemical parameters corresponding to each single cell can be calculated according to a certain electrochemical parameter, and if the statistical characteristics are larger than the preset contrast value of the characteristics, the lithium battery module is determined/judged to have inconsistency.

According to the invention, the method for the consistency analysis of the lithium battery module is based on the microscopic electrochemical parameters to establish the time sequence database of the microscopic electrochemical parameters that are corresponding to each single cell in the lithium battery module, and perform the consistency analysis on each single cell that meets the preset circuit connection relation in the lithium battery module according to the microscopic electrochemical parameters, so as to design a consistency analysis scheme from the perspective of the microscopic electrochemical parameters. Compared the consistency analysis of using macroscopic data such as voltages, temperatures and the like, the novel method is more intrinsic.

In one embodiment, if the microscopic electrochemical parameters include at least one of the SEI film thickness and the total number of active lithium, in the process of acquiring/collecting each microscopic electrochemical parameter corresponding to each single cell in the lithium battery module in step S100, deductive prediction may be performed based on a preset electrochemical life decay model, and, after being combined with the Kalman filtering, maximum likelihood value estimation of the SEI film thickness or the total number of active lithium is performed.

Specifically, the SEI film thickness and the total number of active lithium in the battery are related to the battery aging, logs need to be established, the electrochemical life decay model reflecting the change of the SEI film thickness and the like in the battery is established through mechanism deduction, and the maximum likelihood value estimation is carried out through the Kalman filtering.

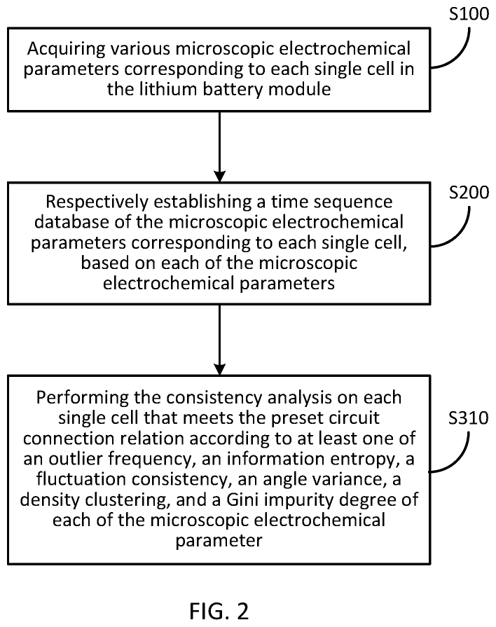

In one embodiment, as shown in FIG. 2, the step S300 performs consistency analysis on each single cell satisfying the preset circuit connection relationship based on each time sequence database, and includes:

At step S310, performing the consistency analysis on each single cell that meets the preset circuit connection relation according to at least one of an outlier frequency, an information entropy, a fluctuation consistency, an angle variance, a density clustering, and a Gini impurity degree of each of the microscopic electrochemical parameter.

Specifically, based on each microscopic electrochemical parameter, at least one of the outlier frequency, the information entropy value, the fluctuation consistency, the angle variance, the density cluster and the Gini impurity degree of the microscopic electrochemical parameter corresponding to each single cell meeting the preset circuit connection relationship is calculated.

In addition to the above statistical parameters, other statistical parameters may also be used in the consistency determination process of the solution.

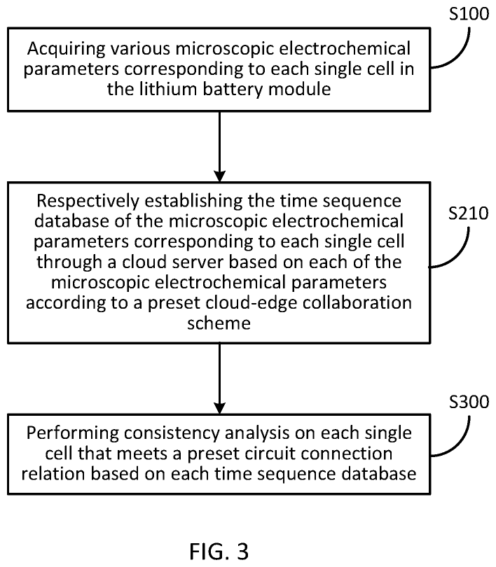

In one embodiment, as shown in FIG. 3, the step S200 of respectively establishing a time sequence database of the microscopic electrochemical parameters corresponding to each single cell, based on each of the microscopic electrochemical parameters includes:

At step S210, respectively establishing the time sequence database of the microscopic electrochemical parameters corresponding to each single cell through a cloud server based on each of the microscopic electrochemical parameters according to a preset cloud-edge collaboration scheme.

Specifically, the scheme adopts a full-order consistency analysis model of various electrochemical parameters, and compared with the prior art that only a macroscopic physical quantity reduced model is adopted, the method is more accurate and predictive, cloud- edge collaboration is realized by adopting a cloud server and a data terminal for realizing the consistency judgment method, repeatedly, the step of establishing a log is not required to be carried out on an integrated circuit on the data terminal for realizing the consistency analysis method, and a time sequence database of the microscopic electrochemical parameters corresponding to each single cell is established to be carried out recording and analysis by the cloud server.

According to the invention, the method for the consistency analysis of the lithium battery module utilizes a cloud server to carry out operations of establishing the time sequence database of the microscopic electrochemical parameters according to the preset cloud-edge collaboration scheme, so that the technical problem of poor consistency analysis effect caused by insufficient hardware capacity or over-low calculation capacity is solved.

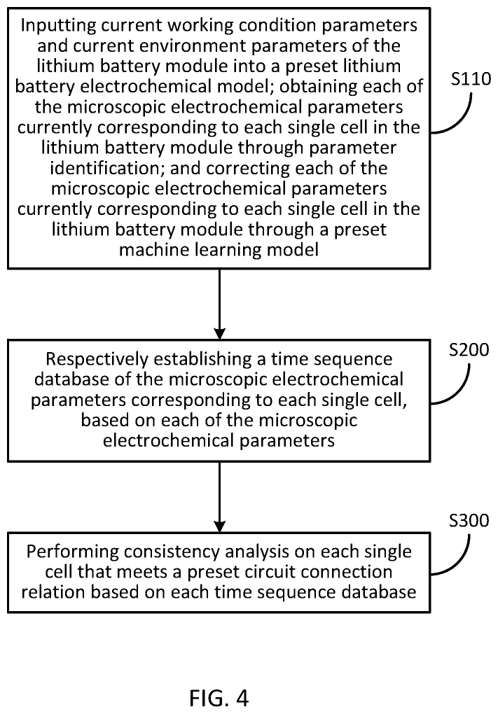

In one embodiment, as shown in FIG. 4, the step S100 of acquiring/collecting each microscopic electrochemical parameter corresponding to each single cell in the lithium battery module specifically includes:

At step S110, inputting current working condition parameters and current environment parameters of the lithium battery module into a preset lithium battery electrochemical model; obtaining each of the microscopic electrochemical parameters currently corresponding to each single cell in the lithium battery module through parameter identification; and correcting each of the microscopic electrochemical parameters currently corresponding to each single cell in the lithium battery module through a preset machine learning model.

Specifically, the electrochemical model of the lithium battery simulates the working state of the battery in the external environment and working condition through the deduction of the material parameters and the electrochemical process of the battery, for example, a common P2D electrochemical model can be adopted, wherein the parameters of the electrochemical model are parameters with real physicochemical meanings.

Further, the machine learning model is generated by machine learning based on working condition parameters and environment parameters of a set of preset sample lithium battery modules and various microscopic electrochemical parameters corresponding to each single cell, the machine learning model compares a battery model simulation result with a real battery result, and corrects the microscopic electrochemical parameters currently corresponding to each single cell in the lithium battery module in a machine learning mode, and the machine learning electrochemical model can be loaded in the cloud server disclosed in the step S210, so that the hardware pressure is further reduced.

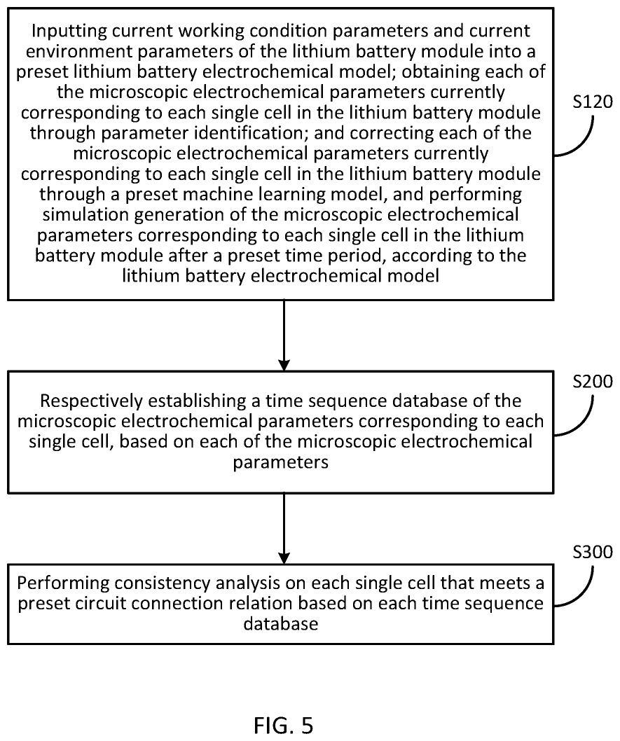

In one embodiment, as shown in FIG. 5, the step S100 of acquiring/colloecting each microscopic electrochemical parameter corresponding to each single cell in the lithium battery module also includes:

At step S120, inputting current working condition parameters and current environment parameters of the lithium battery module into a preset lithium battery electrochemical model; obtaining each of the microscopic electrochemical parameters currently corresponding to each single cell in the lithium battery module through parameter identification; and correcting each of the microscopic electrochemical parameters currently corresponding to each single cell in the lithium battery module through a preset machine learning model, and performing simulation generation of the microscopic electrochemical parameters corresponding to each single cell in the lithium battery module after a preset time period, according to the lithium battery electrochemical model.

In one embodiment, the preset time period is set manually, for example to simulate the electrochemical parameters after 10 hours of operation of the simulation generated cell.

According to the invention, the method for the consistency analysis of the lithium battery module utilizes the lithium battery electrochemical model constructed based on machine learning to predict the trend of the electrochemical parameters of the lithium battery module, and perform the consistency analysis simultaneously according to the present electrochemical parameters and the predicted electrochemical parameters, so that the consistency analysis accuracy of the lithium battery module is improved.

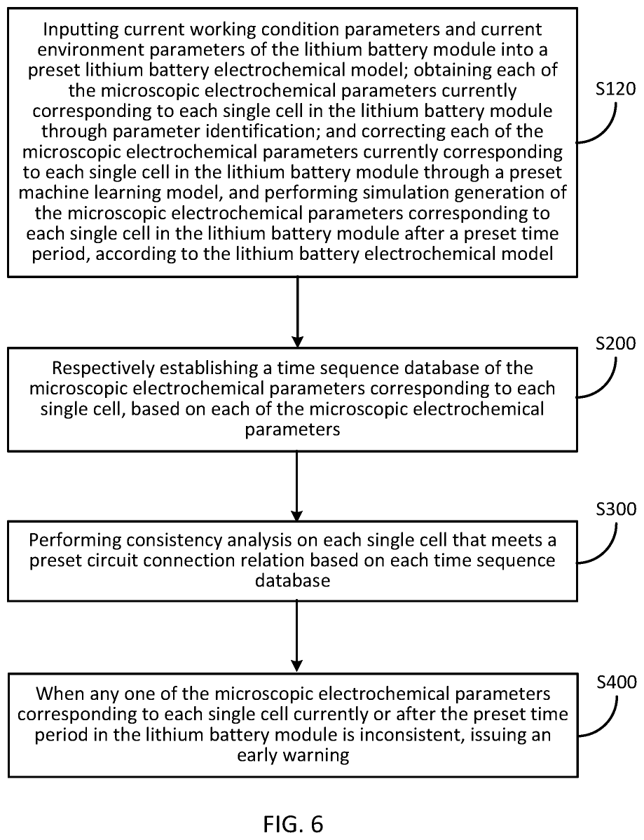

In one embodiment, as shown in FIG. 6, after performing the consistency analysis on each of the battery cells satisfying the preset circuit connection relationship based on each of the time sequence databases, the step S300 further includes:

At step S400, when any one of the microscopic electrochemical parameters corresponding to each single cell currently or after the preset time period in the lithium battery module is inconsistent, issuing an early warning.

According to the invention, the method for the consistency analysis of the lithium battery module utilizes the present electrochemical parameters and the predicted electrochemical parameters to carry out the consistency analysis simultaneously and design an early warning scheme, so that the consistency analysis of the lithium battery module is more predictive.

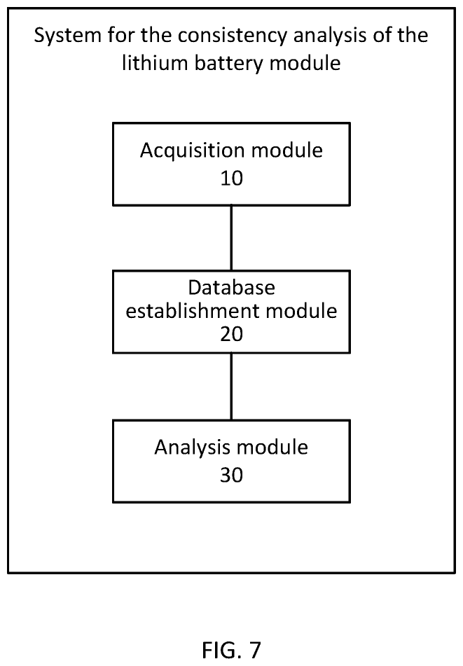

Referring to FIG. 7, the system for the consistency analysis of the lithium battery module is shown according to one embodiment of the invention. The system includes an acquisition module 10, a database establishment module 20, and an analysis module 30.

The acquisition module 10 is configured to acquire various microscopic electrochemical parameters corresponding to each single cell in the lithium battery module.

The database establishment module 20 is connected with the acquisition module 10 and configured to respectively establish a time sequence database of the microscopic electrochemical parameters corresponding to each single cell, based on each of the microscopic electrochemical parameters.

The analysis module 30 is connected with the database establishment module 20 and configured to consistency analysis on each single cell that meets a preset circuit connection relation based on each time sequence database.

Specifically, the microscopic electrochemical parameters are concepts relative to the macroscopic parameters such as voltage, current and temperature in the prior art, and include, but are not limited to, a solid phase diffusion coefficient, a liquid phase diffusion coefficient, a solid phase conductivity, a liquid phase conductivity, sizes of positive and negative electrode regions, a separator region size, an average particle size, a porous medium coefficient, a solid phase volume coefficient, a liquid phase volume coefficient, an electron transfer coefficient, a solid electrolyte interphase (SEI) film thickness, and/or a total number of active lithium. It should be noted that other electrochemical parameters can also be used to practice the invention.

The time sequence database is established for each selected microscopic electrochemical parameter, and the time sequence database comprises time sequence data of the microscopic electrochemical parameter corresponding to each single cell. For example, time sequence data of the solid phase diffusion coefficient corresponding to each of the number 1 to 100 single cells in the lithium battery module is established for the solid phase diffusion coefficient, and is stored in a database corresponding to the solid phase diffusion coefficient, so that the database can be conveniently extracted for consistency analysis in the subsequent process.

The preset circuit connection relationship comprises a series connection, a parallel connection and/or a series-parallel connection, and is determined by manual selection. In the consistency analysis process, the statistical characteristics of the time sequence data of the microscopic electrochemical parameters corresponding to each single cell can be calculated according to a certain electrochemical parameter, and if the statistical characteristics are larger than the preset contrast value of the characteristics, the lithium battery module is determined/judged to have inconsistency.

According to the invention, the system for the consistency analysis of the lithium battery module is based on the microscopic electrochemical parameters to establish the time sequence database of the microscopic electrochemical parameters that are corresponding to each single cell in the lithium battery module, and perform the consistency analysis on each single cell that meets the preset circuit connection relation in the lithium battery module according to the microscopic electrochemical parameters, so as to design a consistency analysis scheme from the perspective of the microscopic electrochemical parameters. Compared the consistency analysis of using macroscopic data such as voltages, temperatures and the like, the novel method is more intrinsic.

In yet another aspect, the invention further provides a non-transitory tangible computer-readable storage medium storing at least one instruction which, when executed by one or more processors, causes the system to perform the method for consistency analysis of the lithium battery module according as disclosed above. For example, the storage medium may be a read-only memory (ROM), a Random Access Memory (RAM), a compact disc read-only memory (CD-ROM), a magnetic tape, a floppy disk, an optical data storage device, and the like.

They may be implemented in program code that is executable by a computing device such that it is executed by the computing device, or separately, or as individual integrated circuit modules, or as a plurality or steps of individual integrated circuit modules. Thus, the invention is not limited to any specific combination of hardware and software.

In the foregoing embodiments, the descriptions of the respective embodiments have their respective emphasis, and reference may be made to the related descriptions of other embodiments for parts that are not described or recited in detail in a certain embodiment.

Those of ordinary skill in the art will appreciate that the various illustrative elements and steps described in connection with the embodiments disclosed herein may be implemented as electronic hardware or combinations of computer software and electronic hardware. Whether such functionality is implemented as hardware or software depends upon the particular application and design constraints imposed on the implementation. Skilled artisans may implement the described functionality in varying ways for each particular application, but such implementation decisions should not be interpreted as causing a departure from the scope of the invention.

In the embodiments provided in the invention, it should be understood that the disclosed lithium battery module consistency analysis method, system and storage medium may be implemented in other manners. For example, the above-described embodiments of a method, a system, and a storage medium for analyzing consistency of a lithium battery module are merely illustrative, for example, the division of the module or the unit is only a logical function division, and there may be another division manner in actual implementation, for example, a plurality of units or modules may be combined or may be integrated into another system, or some features may be omitted, or may not be executed. In another aspect, the communication links shown or discussed with respect to each other may be electrical, mechanical or other forms through some interfaces, device or unit communication links or integrated circuits.

The units described as separate parts may or may not be physically separate, and parts displayed as units may or may not be physical units, may be located in one place, or may be distributed on a plurality of network units. Some or all of the units can be selected according to actual needs to achieve the purpose of the solution of the embodiment.

In addition, functional units in the embodiments of the invention may be integrated into one processing unit, or each unit may exist alone physically, or two or more units are integrated into one unit. The integrated unit can be realized in a form of hardware, and can also be realized in a form of a software functional unit.

It should be noted that the above-mentioned embodiments are only preferred embodiments of the invention, and it should be noted that, for those skilled in the art, various modifications and decorations can be made without departing from the principle of the invention, and these modifications and decorations should also be regarded as the protection scope of the invention.

What is claimed is:

1. A method for consistency analysis of a lithium battery module, comprising:
acquiring various microscopic electrochemical parameters corresponding to each single cell in the lithium battery module;
respectively establishing a time sequence database of the microscopic electrochemical parameters corresponding to each single cell, based on each of the microscopic electrochemical parameters; and
performing consistency analysis on each single cell that meets a preset circuit connection relation based on each time sequence database;
wherein said establishing the time sequence database of the microscopic electrochemical parameters corresponding to each single cell based on each microscopic electrochemical parameter comprises:
respectively establishing the time sequence database of the microscopic electrochemical parameters corresponding to each single cell through a cloud server based on each of the microscopic electrochemical parameters according to a preset cloud-edge collaboration scheme.

2. The method of claim 1, wherein the microscopic electrochemical parameters comprise at least one of a solid phase diffusion coefficient, a liquid phase diffusion coefficient, a solid phase conductivity, a liquid phase conductivity, sizes of positive and negative electrode regions, a separator region size, an average particle size, a porous medium coefficient, a solid phase volume coefficient, a liquid phase volume coefficient, an electron transfer coefficient, a solid electrolyte interphase (SEI) film thickness, and a total number of active lithium.

3. The method of claim 2, wherein the microscopic electrochemical parameters include at least one of the SEI film thickness and the total number of active lithium, and said acquiring the microscopic electrochemical parameters corresponding to each single cell in the lithium battery module comprises:
performing deductive prediction based on a preset electrochemical life decay model, and performing maximum likelihood value estimation of the SEI film thickness or the total number of active lithium after Kalman filtering.

4. The method of claim 1, wherein said performing the consistency analysis on each single cell that meets the preset circuit connection relation based on each time sequence database comprises:
performing the consistency analysis on each single cell that meets the preset circuit connection relation according to at least one of an outlier frequency, an information entropy, a fluctuation consistency, an angle variance, a density clustering, and a Gini impurity degree of each of the microscopic electrochemical parameter.

5. The method of claim 1, wherein said acquiring the microscopic electrochemical parameters corresponding to each single cell in the lithium battery module comprises:
inputting current working condition parameters and current environment parameters of the lithium battery module into a preset lithium battery electrochemical model; obtaining each of the microscopic electrochemical parameters currently corresponding to each single cell in the lithium battery module through parameter identification; and correcting each of the microscopic electrochemical parameters currently corresponding to each single cell in the lithium battery module through a preset machine learning model;
wherein the machine learning model is generated by machine learning, based on the working condition parameters, the environment parameters and the microscopic electrochemical parameters corresponding to each single cell of a set of preset sample lithium battery modules.

6. The method of claim 5, wherein said acquiring the microscopic electrochemical parameter corresponding to each single cell in the lithium battery module further comprises:
generating, by simulation of the lithium battery electrochemical model, each of the microscopic electrochemical parameter corresponding to each single cell in the lithium battery module after a preset time period.

7. The method of claim 6, wherein, after performing consistency analysis on each single cell that meets the preset circuit connection relation based on each time sequence database, the method further comprises:
when any one of the microscopic electrochemical parameters corresponding to each single cell currently or after the preset time period in the lithium battery module is inconsistent, issuing an early warning.

8. A system for consistency analysis of a lithium battery module, comprising:
an acquisition module, configured to acquire various microscopic electrochemical parameters corresponding to each single cell in the lithium battery module;
a database establishment module connected with the acquisition module and configured to respectively establish a time sequence database of the microscopic electrochemical parameters corresponding to each single cell, based on each of the microscopic electrochemical parameters; wherein the database establishing module is configured to respectively establish the time sequence database of the microscopic electrochemical parameters corresponding to each single cell through a cloud server based on each of the microscopic electrochemical parameters according to a preset cloud-edge collaboration scheme; and
an analysis module connected with the database establishment module and configured to consistency analysis on each single cell that meets a preset circuit connection relation based on each time sequence database.

9. A non-transitory tangible computer-readable storage medium storing at least one instruction which, when executed by one or more processors, causes a system to perform the method for consistency analysis of the lithium battery module according to claim 1.
